(19) United States
(12) Patent Application Publication
(10) Pub. No.: US 2009/0026005 A1
(43) Pub. Date: Jan. 29, 2009

(54) Stethoscope warming device with slidable and grip means

(76) Inventor: Raymond Owen West, Belfair, WA (US)

Correspondence Address: RAYMOND O. WEST, PO Box 1137, BELFAIR, WA, 98528, US

(21) Appl. No.: 11/881,387

(22) Filed: Jul. 27, 2007

(51) Int. Cl. A61B 7/02

(52) U.S. Cl. 181/131

(57) ABSTRACT

The present invention describes means for maintaining position of a slidable object on an elongated object such as a flexible tube. The slidable means is provided through paired, generally flattened flexible tension-strips. Likewise, the fixation means is provided through the gripping action of the paired flexible tension-strips.

Immediate and most pertinent use relates to a warmed pouch with slidable attachment to the tube member of a stethoscope. When the tube portion of a stethoscope is inserted and threaded through the lips of the pouch the flexible strips provide friction and grasping. Thus position of the pouch on the tube is maintained in stable fashion until external pressure is applied to the pouch at or near the bilateral ends of the strips.

The present application is a natural improvement of U.S. Pat. No. 5,172,683, issued Dec. 22, 1992.

### FIELD OF INVENTION

This present invention relates generally to slideable objects with fixation means. More specifically, it relates to a slideable warming pouch positioned on a tubular member of a stethoscope. Selectively, it maintains position by means of the grasping action of paired spring-like flexible tension-bands. Position is maintained until said pouch is altered by an outside force.

### BACKGROUND OF INVENTION

It is common knowledge that stethoscopes are used to convey sounds from the cavities of the human body to the ear of the examiner. The sound detecting portion of the stethoscope is usually made of metal, plastic or both. These materials are generally colder than the skin. That temperature gradient causes distress, particularly to infants, children and frail adults. Not uncommonly, the cold object causes withdrawal, squirming, crying and distraction. The possibility of tachycardia, and even other dysrhythmias, exists when the anticipated application of cold causes apprehension and distress.

Others have considered this and have contributed prior art to alleviate the problem. These prior art devices have inherent deficiencies which detract from their utility. A device is disclosed in U.S. Pat. No. 3,766,361 to Swinyar et al. This device embodies an electrically heated holder which is attached to a convenient wall. Thus, this device is not portable, not readily utilized when the examiner must examine individuals in multiple locations.

U.S. Pat. No. 3,213,960 to Wagner discloses a stethoscope-head cover which merely insulates the head member of the stethoscope from ambient temperature change. Without prior warming, it does little to provide warmth to the skin.

Another device, U.S. Pat. No. 4,007,806 to Nobles Jr. employs a heating element mounted in the head member of the stethoscope. Source of heat is provided by battery means. This in turn requires a uniquely constructed stethoscope head. Thus it fails to allow for retrofit with existing stethoscopes.

The present improvement of the stethoscope warmer, subject of U.S. Pat. No. 5,172,683 relates to the pouch member. In its current embodiment the pouch member is formed with an opening on its proximate end. This opening, or aperture, receives the stethoscope head member for the purpose of warming said head of the stethoscope. The pouch member is internally divided roughly in equal halves by a wall member. Said wall member is disposed and secured within the pouch member in a manner forming a first pocket and a second pocket. In this respect the wall member serves to separate the first pocket from the second pocket. In the preferred embodiment, the first pocket is sized and configured to receive the diaphragm and the bell of the stethoscope. The second pocket is sized and configured to receive the heating element. Thus the wall member is preferably constructed from a sheet of heat conducting material which is adapted to promote heat flow from the second pocket which contains the heating element. Thus the sound conducting head of the stethoscope is maintained at a desired temperature when it is inserted into the first pocket. The preferred heating element comprises a packet sized to fit nicely into the second pocket. The warming element includes chemicals which when activated produce an exothermic reaction. Thus the present invention is utilized when the examiner inserts a warming packet into the second pocket. Thereafter the head of the stethoscope is inserted into the first pocket of the pouch, prior to use. When said warming packet no longer produces heat, it is removed from the second pocket and a new packet is inserted.

A Velcro fastener provides closure means for the second pocket when the heating element is in situ.

In accordance with the embodiment of U.S. Pat. No. 5,172,683 the pouch member includes in its distal extremity an opening defining a portal of entry and passage for tube member. This aperture is sized and configured to slidably receive the elongate tubular portion of the stethoscope. When the sound-carrying head of the stethoscope is removed from the end of the tubular portion, the distal extremity of the tubular member may be threaded through this aperture. Thereafter, the pouch member may be selectively slid along the length of the tube member. The head member of said stethoscope is subsequently reattached to the distal end of the tube member. By this means the pouch member is attached to and is carried on the elongated tubing member of the stethoscope.

New and Preferred Embodiment

This application introduces a new embodiment of the sized and configured distal aperture of the pouch member. The embodiment of U.S. Pat. No. 5,172,683 is configured with apertures at the proximal and the distal extremities of the said pouch. In the present embodiment the distal aperture is rounded and sized to approximately fit the circumference of the tubular member of the stethoscope. While this embodiment is functional it has the disadvantage of poor fit if the tubular portion is significantly larger or smaller than the sized circular aperture of the distal extremity of the pouch. Thus selective sliding of the pouch member on the tubular portion can be awkward.

The new embodiment replaces the sized circular aperture presently formed into the distal extremity of the pouch. The new embodiment employs flat paired strips of a flexible substance installed adjacently from side to side on each lip of the distal extremity of said pouch. The paired strips, are bonded or otherwise fastened to the fabric that comprises the pouch. These paired strips are attached, one on each of the two lips of the distal end of the pouch. When not subject to external end to end pressure said strips assume a flat side by side configuration. Thus the aperture is in a closed configuration. When flexed by force applied to the ends of the spring-like pair of flexible strips simultaneously they assume an elliptical configuration. Thereby, the closed distal aperture of said poach is opened to assume said elliptical shape. By this means the pouch can receive the sound-conveying end of the stethoscope along with the tubular portion. In this embodiment, the entire pouch assumes a slidable means on the tubular portion of the stethoscope. Thereafter the pouch member may be selectively slid backward and forward along the tubular portion until the head of the stethoscope is received through the distal aperture to reside within the pouch. Therein the head of the stethoscope is positioned adjacent to the heating portion for warming.

A decided advantage to this embodiment is stasis of the pouch on the tubular portion of the stethoscope. That is, the semi rigid tension-bands, when separated and curved by the presence of the said tubular portion, provide a grasping or clinging effect on the tube. Thereby, the pouch remains fixed on the tube until its position is altered by a significant outside force.

### FURTHER BACKGROUND OF THE INVENTION

This embodiment of paired flexible bands has been employed effectively in prior art.

U.S. Pat. No. 3,063,487, by Mullin employs paired spring-like bands. A change purse construction is described. Further, U.S. Pat. No. 5,069,261 employs resilient walls or flaps to provide facile opening and closure means to provide security for the contents of a coin purse.

In U.S. Pat. No. 3,272,248 O'Ferrell claims a pair of stays for closure, with a long memory, of flexible receptacles. The stays are inherently spring loaded to remain adjacently flat when inactive.

Almog, in U.S. Pat. No. 5,000,500 employs semirigid strips on the lips of a plastic bag. This bag is designed for the sanitary and asthetic collection of dog's excrement and other offensive substances.

Employing similar means, Cailleteau describes a hygienic safety bag fitted with similar flexible materials. This is described in U.S. Pat. No. 5,745,926.

U.S. Pat. No. 6,361,210 by Denco, discloses this embodiment to protect various objects from water damage; when objects are confined in a case.

It is an object of this present invention to provide means for slidable fixation of an object on a rigid or flexible tube and other elongated, rigid or bendable object.

Another object of the present invention is to provide a slidable warming device for stethoscopes. The device embodies an aperture guarded by flexible springlike tension-strips. This affords means for a closed aperture when the said strips are in a flat inactive, closed state. Appropriately, it also provides an elliptical opening when pressure is applied bilaterally to the extremities of the flexible strips.

A third object of this present invention is to provide fixation of the warming pouch on the tubular member of the stethoscope. Said fixation yields to outside force in a slidable fashion.

Further objects and advantages of the present invention will become apparent to those skilled in the prior art. Consideration of the accompanying drawings of the preferred embodiment will facilitate application of this art.

The present invention is not limited to the embodiment as described herein. The grasping or gripping effect of springlike flexible tension-strips when mounted adjacently is unique. Its embodiment is applicable for any device which requires slidable fixation of an object threaded on an elongated object.

### BRIEF DESCRIPTION OF THE DRAWINGS

FIG. 1 is a perspective view of the pouch fitted with paired flexible strips of semirigid springlike substance. Said strips are attached to the distal end of the pouch. Said flexible strips are depicted with flat surfaces adjacent, flat, straight and inactive.

FIG. 2 depicts the flexible strips in the curved, elliptical mode suitable for insertion of a tubular member or an elongated rigid or flexible member of a variety of substances.

FIG. 3 is a perspective view of a tubal portion inserted through a pouch and maintained at station by means of the grasping power of the flexible bands.

FIG. 4 is a perspective view of a slidable object inserted over a solid elongated object.

### DETAILED DESCRIPTION OF THE PREFERRED EMBODIMENT

Referring now to the drawings wherein the presentations are for the purpose of illustrating a preferred embodiment of the present invention only. They are not for purposes of limiting the same.

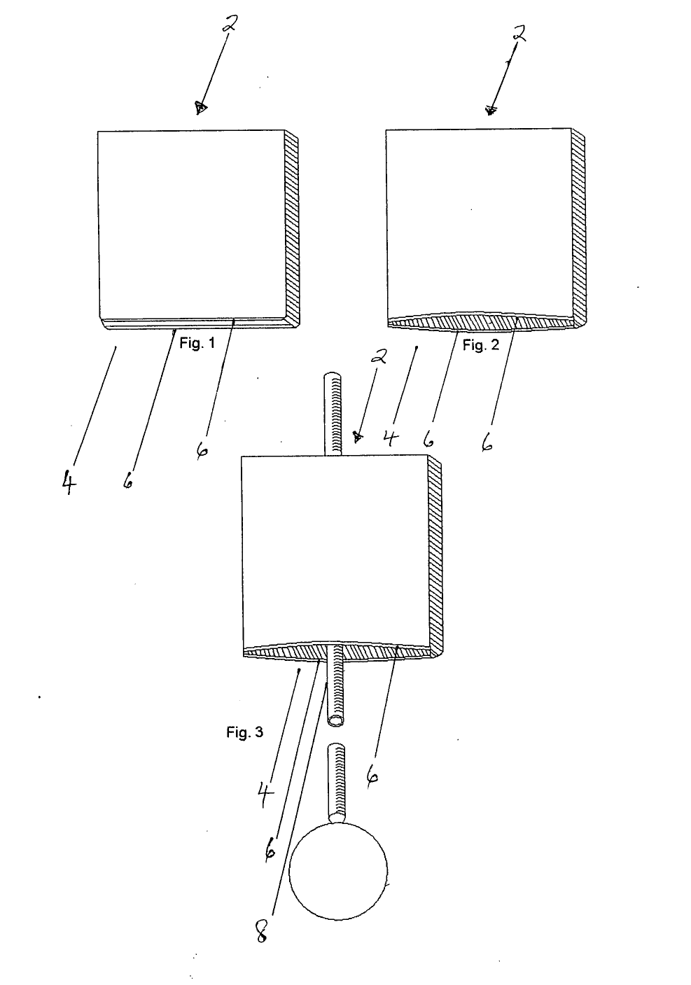

FIG. 1 perspectively illustrates a rectangular object, herein a stethoscope warning device, 2. It is constructed in accordance with the preferred embodiment of the present invention as employed by a provider of medical care.

As shown in FIG. 1 the pouch has a distal extremity 4. Paired flexible tension-strips 6 secure this distal extremity 4.

FIG. 2 demonstrates said flexible strips as they appear in open, elliptical configuration. This configuration obtains when pressure is applied simultaneously to each end of the paired tension-strips.

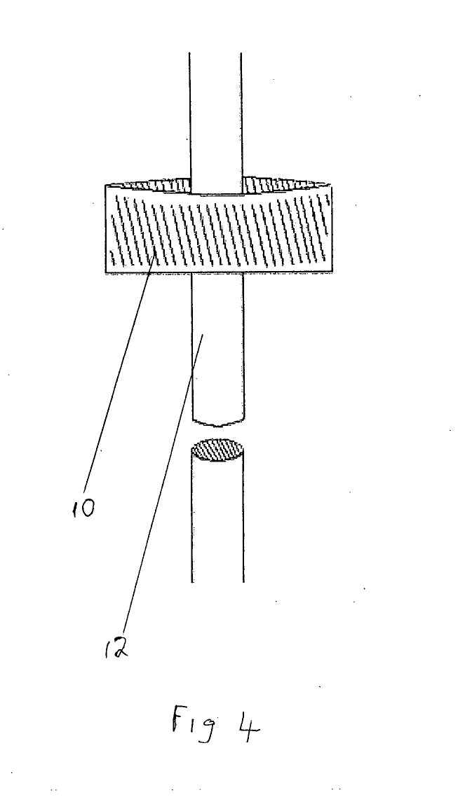

FIG. 3 depicts pouch 2 with tubal member of stethoscope 8 inserted through the length of pouch 2. Flexible strips 6 are hugging or grasping the tubal portion, Thereby they fix the pouch 2 in preferred position.

FIG. 4 depicts a slidable object 10 fitted with flexible strips. Slidable object 10 is inserted, or threaded, on a rigid elongated object 12.

Additional modifications and improvements of the invention may also be apparent to those skilled in the art. Thus the particular combinations of the members described and illustrated herein is intended to represent only two embodiments. They are not intended to serve as limitations of a plethora of alternate devices within the spirit and scope of the invention.

What is claimed:

1. A portable means of slidable fixation of an object inserted on a flexible tube or other elongated object comprising:
paired flexible strips of a variety of bendable substances
paired bendable strips whose flat surfaces are secured adjacently to the flexible lips of a slidable object
paired springlike strips bonded or otherwise secured to the distal extremity of a slidable object
paired bendable strips which gap when pressure is applied bilaterally simultaneously to the ends, thus to form an elliptical opening for entry through the flexible lips of a slidable object on a tube or other elongated object
paired flexible flattened strips which grasp the tube or other elongated object and thereby provide friction and stasis of an object threaded on the tube or other elongated object.
The device of claim 1, wherein a warmed resilient roughly rectangular pouch is employed as a means to maintain warmth to the metal or plastic sound conveying head of a stethoscope comprising
a warmed pouch with flexible tension-strips bonded to its resilient lips
a warmed pouch whose spring steel like strips grasp the tube member of a stethoscope when inserted thereon
a warmed pouch with slidable grasping means when employed on a tubular member of a stethoscope.
